(19) United States
(12) Patent Application Publication
(10) Pub. No.: US 2009/0275703 A1
(43) Pub. Date: Nov. 5, 2009

(54) PROCESS FOR CROSS-LINKING COPOLYMERS USING A SULFUR-CONTAINING CO-AGENT

(75) Inventors: Maria Montserrat Alvarez Grima, Maastricht (NL); Auke Gerardus Talma, Bathmen (NL); Rabindra Nath Datta, Schalkhaar (NL); Jacobus Wilhelmus Noordermeer, Nieuwstadt (NL); Francesca Romana De Risi, Enschede (NL)

Correspondence Address: KENYON & KENYON LLP, ONE BROADWAY, NEW YORK, NY, 10004, US

(73) Assignee: STICHTING DUTCH POLYMER INSTITUTE, Eindhoven (NL)

(21) Appl. No.: 12/158,607

(22) PCT Filed: Dec. 15, 2006
(86) PCT No.: PCT/EP2006/069758

§ 371 Date: Nov. 24, 2008

Related U.S. Application Data
(60) Provisional application No. 60/765,178, filed on Feb. 6, 2006.

(30) Foreign Application Priority Data
Dec. 20, 2005 (EP) 05077943.8

(51) Int. Cl. C07D 207/452; C08F 2/00; C08F 8/00

(52) U.S. Cl. 525/194; 548/521; 526/204

(57) ABSTRACT

Process for cross-linking ethylene-α-olefin copolymers comprising the step of combining the copolymer with (a) an initiator capable of generating free radicals and (b) a sulfur-containing co-agent according to formula R—Y—Sₓ—Y—R. This process results in cross-linked copolymers with improved dynamic properties, tensile strength, delta torque, and/or modulus, without compromising on compression set or aging stability.

The present invention relates to a process to cross-link an ethylene-α-olefin copolymer using a sulfur-containing co-agent. It further relates to the cross-linked copolymer obtainable by this process, and to a new co-agent.

Rubbers are generally cross-linked using either peroxide or sulfur as cross-linking agent. Sulfur is the most common cross-linking agent for the widely used diene-containing elastomers, such as natural rubber, SBR, and polybutadiene; peroxides are the most important cross-linking agents for rubber containing few or no double bonds in the chain, such as EPM and EPDM. In peroxide cross-linking, co-agents are used to improve the cross-link efficiency.

As explained by W. Hofmann, Progress in Rubber and Plastics Technology, vol. 1 (1985), pp. 18-46, the cross-link efficiency of highly unsaturated rubbers, such as styrene-butadiene rubber (SBR), differs from that of less unsaturated rubbers, such as the ethylene-propylene copolymers EPM and EPDM. Within the group of ethylene-propylene rubbers, the efficiency also depends on the amount of ethylene units relative to the amount of propylene units.

Conventionally used co-agents applied in the peroxide cross-linking of ethylene-propylene copolymers are ethylene glycol dimethacrylate (EDMA), and N,N′-m-phenylene bismaleimide (BMI-MP). See W. Hofmann, Progress in Rubber and Plastics Technology, vol. 1 (1985), pp. 18-46, and P. Kovacic and R. W. Hein, J. Am. Chem. Soc., vol. 81 (1958) 1190-1194.

Unfortunately, however, the dynamic and mechanical properties of peroxide vulcanizates obtained when using these co-agents are far from the level obtained by sulfur cross-linking. Further, improvements in, e.g. delta torque, tensile strength, or modulus go generally at the expense of compression set and aging stability.

Surprisingly, it has now been found that the properties of vulcanizates can be improved without compromising on the compression set or aging stability, by using a co-agent with the formula:

R—Y—Sₓ—Y—R (I)

wherein x=1-4, Y is selected from (i) cyclic and acyclic aliphatic groups with 2-20 carbon atoms and (ii) aromatic groups with 6-18 carbon atoms optionally substituted with alkyl groups, and wherein groups (i) and (ii) are optionally substituted with halogen, O, Si, and/or P, and R is selected from:

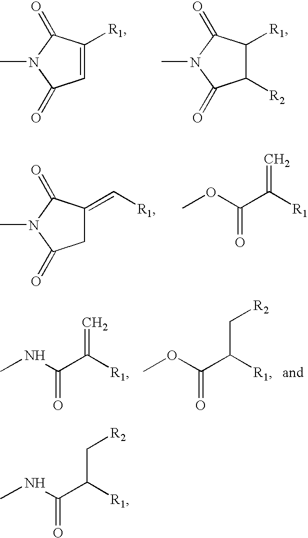

wherein R₁ is selected from hydrogen, halogen, and alkyl groups with 1-18 carbon atoms optionally substituted with hydroxyl, ether, ester, halogen, or sulfur-containing groups, and R₂ is a suitable leaving group.

The present invention therefore provides a process for cross-linking ethylene-α-olefin copolymers comprising the step of combining the copolymer with (a) an initiator capable of generating free radicals and (b) a sulfur-containing co-agent according to formula (I).

This process results in cross-linked ethylene-α-olefin copolymers with improved dynamic properties, tensile strength, delta torque, and/or modulus, without compromising on compression set or aging stability. What is more, the aging stability can be further improved.

The value of x in formula (I) is preferably 1 or 2. R₁ is preferably selected from hydrogen, alkyl groups with 1-4 carbon atoms, CH₂OH, CH₂Cl, CH₂Br, CH₂NH₂, CH₂CN, CH₂COOH, CH₂OR, SO₂R′, SR′, SSR′, CHCl₂, CCl₃, CHBr₂, CBr₃, CH₂F, CF₃, and halogen. More preferably, R₁ is hydrogen or CH₃.

R₂ is a suitable leaving group, i.e. a group or atom that becomes cleaved from the substrate molecule at appropriate reaction rate. Suitable leaving groups include the leaving groups listed in WO 96/20246, page 6, lines 12-22, which is incorporated by reference. Preferred leaving groups are chlorine, bromine, and furan groups.

In a further embodiment, the co-agent is selected from the following compounds:

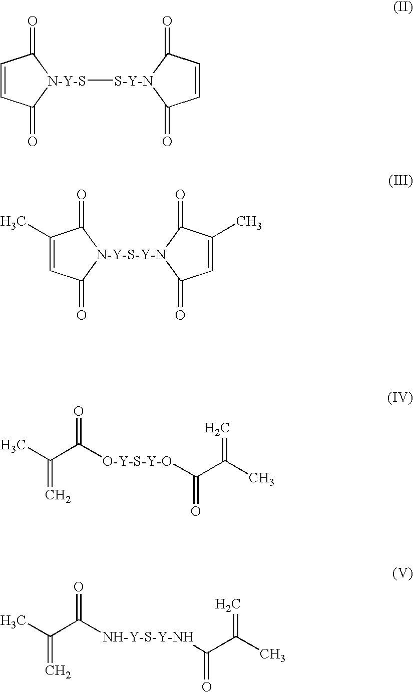

wherein Y is as defined above.

Of these co-agents, the compounds of formulae (III), (IV), and (V) are the more preferred, with the compound of formula (V) being the most preferred.

The degree of unsaturation of the copolymers to be cross-linked by the present process, that is: the amount of diene monomers relative to the total amount of monomers incorporated, is less than 15 wt %, preferably less than 10 wt %, and most preferably less than 5 wt %. This degree of unsaturation can be determined by infrared spectroscopy, using the test recommended by the IISRP working group as described in: Matériaux et Techniques, 1991, p. 69, and referred to by J. W. M. Noordermeer in: Kautschuk Gummi Kunststoffe, vol. 49 (1996), pp. 521-531.

The invention also relates to the co-agent of formula (III) wherein Y is p-phenyl, a compound that has not been disclosed before. This co-agent can be prepared by reacting two parts of citraconic anhydride and one part of H₂N—Y—S—Y—NH₂. For instance, citraconic anhydride is dissolved in an appropriate solvent (e.g. acetic acid). H₂N—Y—S—Y—NH₂ is then added at a temperature in the range of about 60 to 100° C. and the resulting mixture is reacted for 2 to 6 hours at 120 to 160° C. The co-agent is obtained after removal of the solvent by, e.g., evaporation.

Examples of suitable ethylene-α-olefin copolymers that can be cross-linked according to the process of the present invention are ethylene-α-olefin elastomers, ethylene-α-olefin diene elastomers, ethylene-α-olefin block copolymers, ethylene-α-olefin diene block copolymers, and blends thereof. Examples of suitable α-olefins are propylene, butylene, and octene, with propylene being the most preferred. Preferred copolymers are EPM rubbers, EPDM rubbers. If desired, the EPDM rubber may have been blended with polypropylene (PP). Dynamic vulcanisation of a PP/EPDM blend results in the formation of a so-called PP/EPDM theromoplastic vulcanizate (TPV).

The content of ethylene units in the copolymer, relative to the total amount of ethylene and α-olefin units in the copolymer, preferably ranges from 45 to 75 wt %.

The ethylene content can be determined by ASTM Standard Test Method D3900, using method A, as recommended by the IISRP working group and referred to by J. W. M. Noordermeer in: Kautschuk Gummi Kunststoffe, vol. 49 (1996), pp. 521-531.

In formulae (I)-(V), Y is selected from (i) cyclic and acyclic aliphatic groups with 2-20 carbon atoms and (ii) aromatic groups with 6-18 carbon atoms optionally substituted with alkyl groups, and wherein groups (i) and (ii) are optionally substituted with halogen, O, Si, and/or P. Preferably, Y is selected from aliphatic groups with 2-10 carbon atoms, or aromatic groups with 6-12 carbon atoms optionally substituted with alkyl groups. More preferably, Y is selected from acyclic aliphatic groups with 2-4 carbon atoms, and aromatic groups with 6 carbon atoms. Even preferably, Y is a phenyl group. Most preferably, Y is a p-phenyl group.

In the process according to the invention, the amount of co-agent used is preferably at least 1, more preferably at least 4, and even most preferably at least 10 mmole per 100 g copolymer. It is preferably not more than 30, more preferably not more than 22, and most preferably not more than 15 mmole per 100 g copolymer.

The initiator capable of generating free radicals used in the process of the invention is typically selected from compounds with labile C—C, O—O, N—N, O—C bonds, but also can be selected from other products that are precursors for free radicals, e.g., after excitation with radiation. Preferably, it is selected from compounds that are thermally labile, meaning that the free radical is produced upon heating the compound. Preferred thermally labile compounds are C—C initiators, azo-initiators, and peroxides. Preferred C—C initiators are 2,3-dimethyl-2,3-diphenyl butane (Perkadox® 30) and 3,4-dimethyl-3,4-diphenyl hexane (Perkadox 58). Preferred peroxides are perketals, peresters, dialkyl peroxides, diacyl peroxides, trioxepane compounds of the following formula

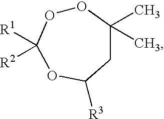

wherein R¹, R², and R³ are independently selected from hydrogen and a substituted or unsubstituted hydrocarbyl group, and cyclic ketone peroxides with a structure represented by the formulae I-III:

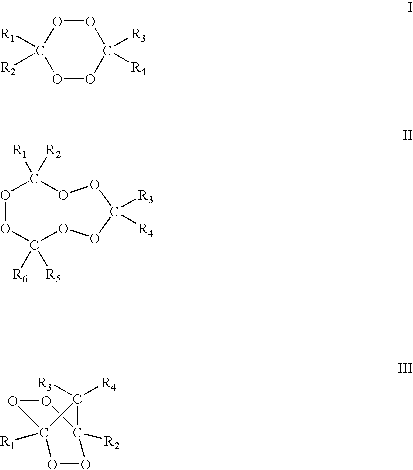

wherein R₁-R₆ are independently selected from the group consisting of hydrogen, C₁-C₂₀ alkyl, C₃-C₂₀ cycloalkyl, C₆-C₂₀ aryl, C₇-C₂₀ aralkyl, and C₇-C₂₀ alkaryl, which groups may include non-cyclic or branched alkyl moieties; and each of R₁-R₆ may optionally be substituted with one or more groups selected from C₁-C₂₀ alkyl, linear or branched, C₃-C₂₀ cycloalkyl, C₆-C₂₀ aryl, C₇-C₂₀ aralkyl, hydroxy, C₁-C₂₀ alkoxy, C₆-C₂₀ aryloxy, C₇-C₂₀ aralkoxy, C₇-C₂₀ alkaryloxy, R₁C(O)O—, R₁OC(O)—, halogen, carboxy, nitrile, and amido; or R₁/R₂, R₃/R₄ and R₅/R₆ may each, together with the carbon atom to which they are attached, form a 3 to 20 atom-membered cycloaliphatic ring which may optionally be substituted with one or more groups selected from C₁-C₂₀ alkyl, non-cyclic or branched, C₃-C₂₀ cycloalkyl, C₆-C₂₀ aryl, C₇-C₂₀ aralkyl, hydroxy, C₁-C₂₀ alkoxy, C₆-C₂₀ aryloxy, C₇-C₂₀ aralkoxy, C₇-C₂₀ alkaryloxy, R₁C(O)O—, R₁OC(O)—, halogen, carboxy, nitrile, and amido.

Preferred azo-initiators and peroxides are those that have a half life of more than 1 hour at 100° C.

More preferred initiators are peroxides selected from: dialkyl peroxides, such as di-cumyl peroxide (Perkadox BC), t-butyl cumyl peroxide (Trigonox® T), di-t-butyl peroxide (Trigonox® B), di(t-butyl peroxyisopropyl)benzene in the meta, para or mixed meta/para form (Perkadox 14), 2,5-dimethyl 2,5-di(t-butyl peroxy) hexane (Trigonox® 101), 2,5 dimethyl 2,5-di(t-butyl peroxy) hexyne-3 (Trigonox® 145), t-butyl isopropyl cumyl peroxide, di-t-amyl peroxide (Trigonox® 201), and cumyl isopropyl cumyl peroxide; peroxy esters, such as: di(t-butyl peroxy)phthalate, t-butyl peroxy benzoate (Trigonox® C), t-butyl peroxy acetate (Trigonox® F), t-butyl peroxy isopropyl carbonate (Trigonox® BPIC), t-butyl peroxy-2-methyl benzoate (Trigonox® 97), t-butyl peroxy laurate, t-butyl peroxy diethyl acetate (Trigonox® 27), t-butyl peroxy isobutyrate (Trigonox® 41), t-butyl peroxy-3,5,5-trimethylhexanoate (Trigonox® 42), t-amyl peroxy benzoate (Trigonox® 127), and t-amyl peroxy acetate; peroxy ketals, such as ethyl 3,3-di(t-butyl peroxy) butyrate, ethyl 3,3-di(t-amyl peroxy) butyrate, n-butyl 4,4-di(t-butyl peroxy) valerate, 2,2-di(t-amyl peroxy) propane, 2,2-di(t-butyl peroxy) butane (Trigonox® D), 1,1-di(t-butyl peroxy)cyclohexane (Trigonox® 22), 1,1-di(t-butyl peroxy)-3,5,5-trimethyl cyclohexane (Trigonox® 29), and 1,1-di(t-amyl peroxy)cyclohexane (Trigonox® 122); trioxepanes, such as 3,3,5,7,7-pentamethyl-1,2,4-trioxepan (Trigonox® 311); and cyclic ketone peroxides, such as 3,6,9-triethyl-3,6,9-trimethyl-1,4,7-triperoxononane (Trigonox® 301).

The amount of initiator to be used in the process according to the present invention and present in the composition according to the invention, based on the weight of the copolymer, preferably ranges from 0.1, preferably from 1.0, more preferably from 2.0 parts by weight, up to 8, preferably up to 6, most preferably up to 4 parts by weight.

Conventional rubber additives may also be employed, in their usual amounts. For example, reinforcing agents or fillers such as carbon black, silica, clay, chalk, talc, aluminium hydroxide, magnesium hydroxide, and calcium carbonate may be included in the copolymer composition. Other additives such as lubricants, tackifiers, waxes, antioxidants, pigments, UV-stabilization agents, blowing agents, nucleating agents, acceleratores, sulfur, ZnO, extender oils, e.g. paraffinic oils like stearic acid, voltage stabilizers, water free retardants, metal deactivators, coupling agents, dyes, and colorants may also be included alone or in combination. If used, such additives are to be used in an amount sufficient to give the intended effect.

Additives that should preferably not be employed, in particular not when using bis-maleimide type co-agents like the co-agent of formula (II), are antiozonants, S-containing compounds that accelerate sulfur vulcanization, and polysulfide polymers. Such additives may negatively influence the properties of the resulting cross-linked polymer.

According to one embodiment, the copolymer is cross-linked by first thoroughly mixing the copolymer to be cross-linked with initiator, co-agent, and optional additives, followed by a cross-linking step. According to this embodiment, the copolymer to be cross-linked, the co-agent, and the initiator are to be mixed thoroughly without cross-linking occurring. If the initiator is a thermally labile compound, this means that mixing is typically done at temperatures where the half life of the initiator is more than 0.5 hour, preferably more than 1 hour, even more preferably more than 2 hours. In practice, the temperature of the copolymer is limited to 50 to 150° C. during the mixing phase. The mixing can be achieved in various ways, as is known to the skilled person. For instance, the components may be milled on a variety of apparatus including multi-roll mills, screw mills, continuous mixers, compounding extruders, and Banbury mixers, or dissolved in mutual or compatible solvents. When all of the solid components are available in the form of a powder, or as small particles, the components my first be blended in, e.g., a Banbury mixer or a continuous extruder; the blend can then be masticated on a heated mill, for instance a two-roll mill, and milling may be continued until an intimate mixture of the components is obtained. Alternatively, a master batch containing the copolymer and one or more antioxidants may be combined with the initiator and the co-agent.

Where the copolymer is not available in the powder form, the copolymer can be introduced into a mill, masticated until it forms a band around one roll (i.e. a mixing bank), after which the remaining components, either as blend or each of them separate, are added and the milling continued until an intimate mixture is obtained. The rolls are preferably kept at a temperature which is in the range of about 25 to 150° C., more preferably 50-120° C., and is below the rapid decomposition temperatures of the initiator component. The resulting mixture is removed from the mill in the form of a sheet.

After mixing, the copolymer is cross-linked upon the formation of free radicals by the initiator in conventional ways. Preferably, the copolymer is cross-linked at temperatures from 80° C., more preferably 120° C., most preferably 140° C. up to 300° C., more preferably up to 200° C. in a period of 2 minutes up to 2 hours. The most common cross-linking temperatures are in the range of 160-200° C.

For example, all of the components (copolymer, co-agent, initiator, and optional additives) can be blended or compounded together prior to their introduction into an extrusion apparatus from which they are to be extruded at temperatures of about 120 to 160° C. for polyolefins. After being extruded, the copolymer is cross-linked at elevated temperatures of about 140° C. or higher, preferably about 180 to 200° C., using conventional cross-linking procedures.

According to another embodiment, the process of the present invention is a dynamic vulcanization process, wherein the cross-linking reaction is performed during melt-mixing of the copolymer (e.g. EPDM) with a second polymer (e.g. PP). Initiator and co-agent are added during said melt mixing. Suitable mixing equipment includes multi-roll mills, screw mills, continuous mixers, compounding extruders, and Banbury mixers. The temperature during dynamic vulcanization preferably ranges from 80° C., more preferably 120° C., more preferably 140° C., and most preferably 160° C., up to 300° C., more preferably up to 200° C. Dynamic vulcanization generally takes about 1 minute to 1 hour, more preferably 2 to 20 minutes.

This process is especially suitable for the preparation of PP/EPDM theromoplastic vulcanizates (TPVs),

The process according to the invention is preferably performed in a closed system, not in open air, in order to reduce air-inhibition and, hence, to reduce surface tack.

The cross-linked copolymer can have various uses including, without limitation: in tire compositions, such as tread, undertread, sidewall, wire skim, inner liner, and in bead compounds; in industrial rubber compositions, such as hoses, belts, tubes, engine mounts, shock absorbers and isolators, in weather stripping, mouldings, and vehicle bumpers; and in wire and cable, such as semi-conductor and insulating compounds.

### EXAMPLES

### Examples 1-3 and Comparative Examples A and B

Mixing: For all copolymer compositions being evaluated, first a masterbatch comprising 100 parts by weight (pbw) EPM (ethylene content 56 mole %), 60 phr carbon black, 1 phr stearic acid, and 45 phr paraffinic oil was prepared in a conventional way. Dicumyl peroxide (2.4 phr) and co-agent (15 mmol/phr) were added to this masterbatch on a Schwabenthan two-roll mill (friction 1:1.22, temperature 50-70° C., mixing time 10 minutes).

Cross-linking evaluation: The cross-linking was evaluated using a RPA 2000 from Alpha Technologies to determine: the delta torque or extent of cross-linking, being the maximum torque (MH) minus the minimum torque (ML); scorch safety (Ts2), being the time to reach 2dNm above minimum torque (ML); and optimum cure time (t90), being the time to reach 90% of the delta torque above minimum in accordance with method ISO 6502 (Rubber-Measurement of vulcanization characteristics with rotorless curemeters).

Samples for mechanical testing of cross-linked copolymer: Samples were prepared by cross-linking copolymer compositions by compression moulding in a Wickert press at 160° C. for a period of time which was twice the t90 time. The samples were 2 mm thick, in accordance with method ISO 37.

Evaluation of cross-linked copolymer: The cross-linked copolymer was die-cut into dumb-bells and tested in a Zwick tensile tester, in accordance with ISO 37.

The co-agents used in the different examples are indicated in Table 1. Their effect on the Delta Torque, tensile strength, modulus 100, modules 200, compression set, and the shore hardness of the resulting cross-linked copolymer are indicated in Table 2. The development of these parameters during aging at 150° C. is indicated in Table 3.

TABLE 1

| Example no. | Co-agent |
|---|---|
| A (comp.) | N—N′-m-phenylene dimaleimide (BMI-MP) |
| B (comp.) | Ethylene glycol dimethacrylate (EDMA) |
| 1 | Formula (II) with Y = p-phenyl (BMI-PPS2) |
| 2 | Formula (III) with Y = p-phenyl (BCI-PPS1) |
| 3 | Formula (V) with Y = p-phenyl (BMA-PPS1) |

TABLE 2

| | | Tensile | Compression | | | | |
|---|---|---|---|---|---|---|---|
| | Delta Torque | Strength. | set | | Mod100 | Mod200 | Shore A |
| Co-agent | (dNm) | (MPa) | 23° C. | 70° C. | (Mpa) | (Mpa) | hardness |
| BMI-MP | 3.0 | 7.9 | 16 | 22 | 1.2 | 2.7 | 43 |
| EDMA | 2.3 | 7.2 | 20 | 37 | 0.8 | 1.4 | 37 |
| BMI-PPS2 | 5.0 | 8.2 | 13 | 20 | 1.3 | 3.0 | 52 |
| BCI-PPS1 | 4.5 | 9.3 | 15 | 18 | 1.4 | 3.1 | 51 |
| BMA-PPS1 | 3.5 | 8.2 | 17 | 24 | 1.2 | 2.4 | 45 |

TABLE 3

| | Aging time | Tensile Strength. | Mod100 | Mod200 |
|---|---|---|---|---|
| Co-agent | (days) | (MPa) | (MPa) | (MPa) |
| BMI-MP | 0 | 7.9 | 1.2 | 2.7 |
| | 3 | 6.1 | 1.1 | 2.3 |
| | 5 | 4.4 | 1.0 | 2.2 |
| | 7 | 3.6 | 1.1 | 2.2 |
| | 10 | 1.9 | 1.1 | 2.2 |
| BMI-PPS2 | 0 | 8.2 | 1.3 | 3.0 |
| | 3 | 6.1 | 1.3 | 2.8 |
| | 5 | 4.5 | 1.2 | 2.5 |
| | 7 | 3.9 | 1.3 | 2.7 |
| | 10 | 3.5 | 2.0 | n.a. |
| EDMA | 0 | 7.2 | 0.8 | 1.4 |
| | 3 | 5.9 | 0.8 | 1.1 |
| | 5 | 4.8 | 0.8 | 1.0 |
| | 7 | 3.0 | 0.7 | 0.9 |
| | 10 | 1.7 | 0.7 | 0.9 |
| BMA-PPS1 | 0 | 8.2 | 1.2 | 2.4 |
| | 3 | 7.2 | 1.2 | 2.3 |
| | 5 | 6.3 | 1.3 | 2.3 |
| | 7 | 5.6 | 1.2 | 2.2 |
| | 10 | 4.7 | 1.3 | 2.2 |

These data show that the process according to the invention results in cross-linked ethylene-α-olefin copolymer with improved tensile strength, delta torque, modulus, and aging stability. Also the compression set remains satisfactory.

### Examples 4-6 and Comparative Example C

The following experiments serve to illustrate that the process of the present invention allows the cross-linking of EPDM in a blend with PP using dynamic vulcanization. This results in PP/EPDM thermoplastic vulcanizates with satisfying properties. This is surprising, since, according to A. Y. Coran (in: Themoplastic Elastomers, Eds.: N. R. Legge, G. Holden, and H. E. Schroeder, Hanser Publishers, Munich, 1987, chapter 7), the use of peroxides for preparing such vulcanizates should be avoided, mainly because peroxides promote the degradation of PP.

In a Brabender Plasticorder PL2000 internal mixer, 200 phr EPDM (containing 50 wt % paraffinic oil; ethylene content: 63 wt %) was mixed with 43 phr polypropylene (PP) at a set temperature of the mixer of 170° C., and with rotor speed 80 rpm. The mixer chamber was first fed with PP, followed by the stabilizers Irganox® 1076 and Irgafos® 168 and the EPDM one minute later. Mixing was continued for 3 more minutes, after which optionally co-agent (Examples 4-6) was added, followed by the dicumyl peroxide (6.76 phr) after the fifth minute of mixing. Mixing was continued for another 5 minutes. The mixture was subsequently sheeted out on a two-roll mill.

The co-agent used was 4,4-thiobis-(phenyl-methacrylate), which is the co-agent of formula IV, wherein Y is p-phenyl. Comparative Example C was performed in the absence of co-agent.

The resulting rubbers were tested according to the methods explained in relation to the former Examples. The results are displayed in Table 4.

TABLE 4

| | Amount | | | | | Elon- |
|---|---|---|---|---|---|---|
| | co- | Tensile | | | Young's | ation |
| | agent | Strength | Compression set | | Modulus | at break |
| Example | (phr) | (MPa) | 23° C. | 70° C. | (Mpa) | (%) |
| C | — | 3.8 | 27 | 32 | 9.3 | 280 |
| 4 | 2.12 | 5.0 | 20 | 29 | 10.9 | 410 |
| 5 | 4.25 | 6.5 | 21 | 32 | 11.7 | 520 |
| 6 | 6.38 | 5.6 | 22 | 32 | 11.3 | 460 |

These results show that the process according to the invention, due to the presence of the S-containing co-agent, allows the dynamic vulcanization of EPDM in 30/70 PP/EPDM blends, resulting in a TPV with satisfactory properties.

Further experiments have show that the same positive effect of the S-containing co-agent is obtained with 50/50 PP/EPDM and 70/30 PP/EPDM blends.

Also compared to another co-agent, zinc dimethacrylate, did the S-containing co-agent result in improved properties: higher elongation at break, improved compression set (especially at 70° C.), and comparable tensile strength and Young's modulus.

Without wishing to be bound by theory, it is believed that the positive effect of the S-containing co-agent is due to a stabilizing effect on the peroxide-induced polypropylene degradation. Experiments have shown that, compared to the absence of co-agent and the use of ethylene dimethacrylate, zinc diacrylate, and zinc dimethacrylate, the use of the S-containing co-agent in accordance with the invention reduces polypropylene degradation.

1. A process to cross-link an ethylene-α-olefin copolymer comprising the step of combining the copolymer with (a) an initiator capable of generating free radicals and (b) a co-agent with the formula
R—Y—Sₓ—Y—R (I)
wherein x=1-4, Y is selected from the group consisting of (i) cyclic and acyclic aliphatic groups with 2-20 carbon atoms and (ii) aromatic groups with 6-18 carbon atoms optionally substituted with alkyl groups, and wherein groups (i) and (ii) are optionally substituted with at least one of a halogen, O, Si, and P, and R is selected from the group consisting of: 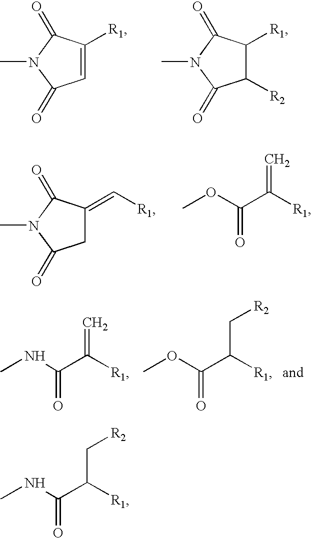
wherein R₁ is selected from the group consisting of hydrogen, halogen, and alkyl groups with 1-18 carbon atoms optionally substituted with hydroxyl, ether, ester, halogen, or sulfur-containing groups, and R₂ is a suitable leaving group.

2. The process according to claim 1 wherein x is 1 or 2.

3. The process according to claim 2 wherein the co-agent is selected from the following compounds: 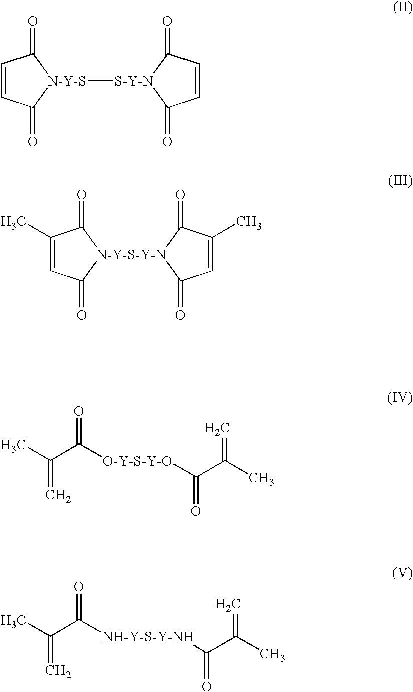
wherein Y is selected from the group consisting of (i) cyclic and acyclic aliphatic groups with 2-20 carbon atoms and (ii) aromatic groups with 6-18 carbon atoms optionally substituted with alkyl groups, and wherein groups (i) and (ii) are optionally substituted with at least one of a halogen, O, Si, and P.

4. The process according to claim 1 wherein the copolymer is selected from the group consisting of ethylene-α-olefin elastomers, ethylene-α-olefin diene elastomers, ethylene-α-olefin block copolymers, ethylene-α-olefin diene block copolymers, and blends thereof.

5. The process according to claim 4 wherein the copolymer is ethylene propylene elastomer (EPM), ethylene-propylene diene elastomer (EPDM), or a blend thereof.

6. The process according to claim 5 wherein the copolymer is ethylene-propylene diene elastomer (EPDM) and wherein the ethylene-propylene diene elastomer (EPDM) is cross-linked in a blend with polypropylene (PP), resulting in a PP/EPDM thermoplastic vulcanizate (TPV).

7. The process according to claim 1 wherein Y is selected from the group consisting of acyclic aliphatic groups with 2-4 carbon atoms, and aromatic groups with 6 carbon atoms.

8. The process according to claim 1 wherein the initiator is selected from the group of compounds with labile C—C, O—O, N—N, and O—C bonds, and mixtures thereof.

9. A bis-citraconimide compound with the formula:

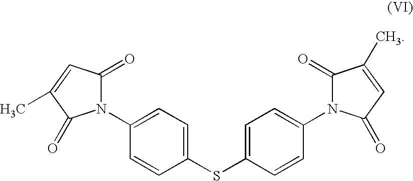

10. A composition comprising (i) an ethylene-α-olefin copolymer, (ii) an initiator capable of generating free radicals, and (iii) a co-agent with the formula
R—Y—Sₓ—Y—R (I)
wherein x=1-4, Y is selected from the group consisting of (i) cyclic and acyclic aliphatic groups with 2-20 carbon atoms and (ii) aromatic groups with 6-18 carbon atoms optionally substituted with alkyl groups, and wherein groups (i) and (ii) are optionally substituted with at least one of a halogen, O, Si, and P, and R is selected from the group consisting of: 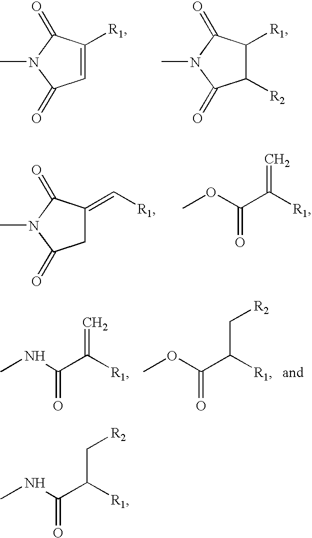
wherein R₁ is selected from the group consisting of hydrogen, halogen, and alkyl groups with 1-18 carbon atoms optionally substituted with hydroxyl, ether, ester, halogen, or sulfur-containing groups, and R₂ is a suitable leaving group.

11. A cross-linked ethylene-α-olefin copolymer obtained by the process of claim 1.

12. The process according to claim 3 wherein the copolymer is selected from the group consisting of ethylene-α-olefin elastomers, ethylene-α-olefin diene elastomers, ethylene-α-olefin block copolymers, ethylene-α-olefin diene block copolymers, and blends thereof.

13. The process according to claim 12 wherein the copolymer is ethylene propylene elastomer (EPM), ethylene-propylene diene elastomer (EPDM), or a blend thereof.

14. The process according to claim 13 wherein the copolymer is ethylene-propylene diene elastomer (EPDM) and wherein the ethylene-propylene diene elastomer (EPDM) is cross-linked in a blend with polypropylene (PP), resulting in a PP/EPDM thermoplastic vulcanizate (TPV).

15. The process according to claim 3 wherein Y is selected from the group consisting of acyclic aliphatic groups with 2-4 carbon atoms, and aromatic groups with 6 carbon atoms.

16. The process according to claim 3 wherein the initiator is selected from the group of compounds with labile C—C, O—O, N—N, and O—C bonds, and mixtures thereof.

17. The composition according to claim 10 wherein the co-agent is selected from the following compounds: 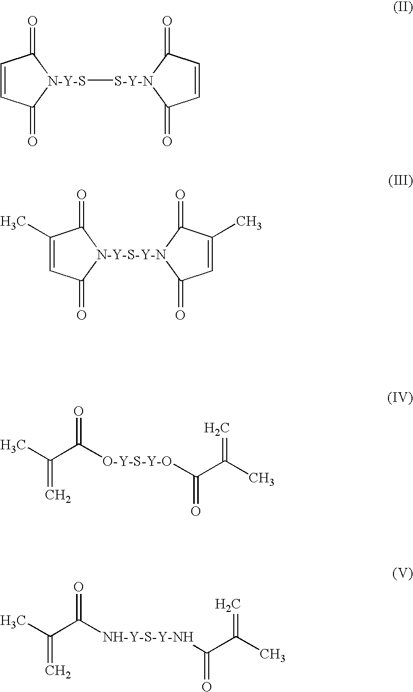
wherein Y is selected from the group consisting of (i) cyclic and acyclic aliphatic groups with 2-20 carbon atoms and (ii) aromatic groups with 6-carbon atoms optionally substituted with alkyl groups, and wherein groups (i) and (ii) are optionally substituted with at least one of a halogen, O, Si, and P.

18. A composition comprising (i) an ethylene-α-olefin copolymer, (ii) an initiator capable of generating free radicals, and (iii) a co-agent with the formula:

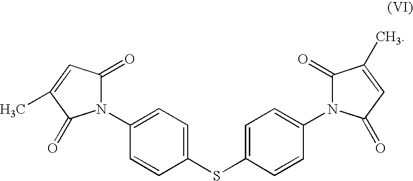

19. The composition according to claim 10 wherein the copolymer is selected from the group consisting of ethylene-α-olefin elastomers, ethylene-α-olefin diene elastomers, ethylene-α-olefin block copolymers, ethylene-α-olefin diene block copolymers, and blends thereof.

20. The composition according to claim 19 wherein the copolymer is ethylene propylene elastomer (EPM), ethylene-propylene diene elastomer (EPDM), or a blend thereof.
